(19) United States
(12) Patent Application Publication
(10) Pub. No.: US 2026/0363540 A1
(43) Pub. Date: Nov. 19, 2026

(54) PUPILLARY DISTANCE DETERMINATION FROM IMAGES INCLUDING REFERENCE FEATURES

(71) Applicant: Walmart Apollo, LLC, Bentonville, AR (US)

(72) Inventors: Elad Laks, Tel Aviv (IL); Yonatan Blum, Givatayim (IL); Geva Bidner, Modi'in (IL); Ofer Saban, Kidron (IL)

(21) Appl. No.: 19/211,767

(22) Filed: May 19, 2025

(51) Int. Cl. A61B 3/11; G02C 13/00; G06T 19/00

(52) U.S. Cl. CPC A61B 3/111; G02C 13/005; G06T 19/006

(57) ABSTRACT

Systems and method for determining pupillary distances. Some examples include identifying dimensions of eyewear worn by a subject of an image and detecting, from the image, a first pupil and a second pupil of the subject. Examples further include determining a pixel distance between the first pupil and the second pupil and converting the pixel distance to a measured distance using the eyewear dimensions, wherein the measured distance is on a lens plane associated with lenses of the eyewear. Examples further include determining a location of an eye plane associated with the eyes of the subject relative to the position of the lens plane using a vertex value and calculating a pupillary distance (PD) using the measured distance on the lens plane, wherein the calculated PD is the distance between the first pupil and the second pupil on the eye plane.

### BACKGROUND

Measuring the pupillary distance (PD) of a subject is an important step in various ophthalmological and optometric applications. For example, the effectiveness of a subject's eyeglasses prescription can directly depend on the accuracy of the subject's PD measurement. Traditionally, PDs are measured by specially trained practitioners, either manually with the use of a ruler, or by employing a specialized piece of medical equipment called a pupilometer, for example.

### SUMMARY

The disclosed examples are described in detail below with reference to the accompanying drawing figures listed below. The following summary is provided to illustrate some examples disclosed herein.

Disclosed are various systems and methods for measuring pupillary distances. Examples of this disclosure include identifying dimensions of eyewear worn by a subject of an image and detecting, from the image, a first pupil and a second pupil of the subject. Examples include determining a pixel distance between the first pupil and the second pupil and converting the pixel distance to a measured distance using the eyewear dimensions, The measured distance is on a lens plane associated with lenses of the eyewear. Examples further include determining a location of an eye plane associated with the eyes of the subject relative to the position of the lens plane using a vertex value and calculating a PD using the measured distance on the lens plane, wherein the calculated PD is the distance between the first pupil and the second pupil on the eye plane.

### BRIEF DESCRIPTION OF THE DRAWINGS

The disclosed examples are described in detail below with reference to the accompanying drawing figures listed below:

FIG. 1 is a block diagram illustrating an example system for determining pupillary distances from images by using eyewear as a reference;

FIG. 2 is a block diagram illustrating another example system for determining pupillary distances from images by using eyewear as a reference;

FIG. 3. is a block diagram illustrating example retrieval of eyewear dimensions from a dimension database based on recognizing eyewear in an image;

FIGS. 4 and 5 are diagrams illustrating example pixel measurements taken from an image of a subject wearing eyewear;

FIG. 6 is a block diagram illustrating an example of how the eyewear dimensions of FIG. 3 are used with the pixel measurements of FIG. 2 to perform pixel-to-millimeter conversion;

FIG. 7 is an illustrative side profile of an example subject having a lens plane associated with lenses of eyewear and an eye plane associated with the eyes of the subject;

FIG. 8 illustrates an example projection of the distance between the pupils on the eye plane compared to the lens plane;

FIG. 9 is a block diagram illustrating an example of how the pupillary distance between the pupils on the eye plane and other associated information is calculated from the distance information measured on the lens plane;

FIG. 10 is a flowchart illustrating an example method for determining a pupillary distance for a subject using eyewear as a refence from an image, such as by using the system of FIG. 1 or the system of FIG. 2, for example; and

FIG. 11 is a flowchart illustrating an example method for performing pixel-to-millimeter conversions, such as for methods for determining a pupillary distance for a subject using eyewear as a refence from an image, such as the method of FIG. 10, for example.

Corresponding reference characters indicate corresponding parts throughout the drawings.

### DETAILED DESCRIPTION

A more detailed understanding can be obtained from the following description, presented by way of example, in conjunction with the accompanying drawings. The entities, connections, arrangements, and the like that are depicted in, and in connection with the various figures, are presented by way of example and not by way of limitation. As such, any and all statements or other indications as to what a particular figure depicts, what a particular element or entity in a particular figure is or has, and any and all similar statements, that can in isolation and out of context be read as absolute and therefore limiting, can only properly be read as being constructively preceded by a clause such as “In at least some embodiments, . . . ” For brevity and clarity of presentation, this implied leading clause is not repeated ad nauseum.

Pupillary Distance (PD) measurements are a vital step in various ophthalmological and optometric applications. Traditionally, PDs are measured by specially trained practitioners, either manually with the use of a ruler, or by employing a specialized piece of medical equipment called a pupilometer. Manual measurements are often inaccurate, and only specially trained practitioners are generally able to use pupilometers effectively. Accordingly, conventional PD measurements have been known to be inaccessible and/or inaccurate.

Aspects of the disclosure solve multiple problems that are necessarily rooted in computer technology, and render use of computing platforms more efficient by providing improved systems and methods for determining PDs using computer vision technology and techniques. Specifically, some embodiments provide a computing device that identifies dimensions of eyewear worn by a subject of an image and detect, from the image, a first pupil and a second pupil of the subject. The computing device determines a pixel distance between the first pupil and the second pupil and converts the pixel distance to a measured distance using the eyewear dimensions, where the measured distance is on a lens plane associated with lenses of the eyewear. The computing device determines a location of an eye plane associated with the eyes of the subject relative to the position of the lens plane using a vertex value and calculates a PD for the subject using the measured distance on the lens plane, wherein the calculated PD is the distance between the first pupil and the second pupil on the eye plane. In some embodiments, the PD is calculated with the use of a machine learning (ML) model.

According to various embodiments, the computing device uses the eyewear dimensions as a reference for determining the PD. Accordingly, a feature within the image is used for performing pixel-to-millimeter conversions in converting the detected PD in pixels to millimeters, thereby allowing for efficient use of in-image features in performing pixel-to-millimeter conversions.

According to various embodiments, the computing device recognizes the eyewear in the image and retrieves the eyewear dimensions from a dimensions database based on the recognition. Thus, this object recognition within image provides for efficient use of in-image features in performing pixel-to-millimeter conversions.

According to various embodiments, the computing device converts a distance determined on a lens plane associated with the lenses of eyewear to a PD on an eye plane associated with the front of the subject's eyes, thereby providing improved accuracy in performing PD calculations.

According to various embodiments, the computing device uses known and highly precise dimension information associated with eyewear in performing pixel-to-millimeter conversion in calculating the subject's PD, thereby providing improved accuracy in performing PD calculations.

According to various embodiments, the computing device uses multiple pixel distances taken from the image in performing pixel-to-millimeter conversion to account for overlapping and/or undetectable features of the eyewear, thereby providing improved accuracy in performing the pixel-to-millimeter conversion and ultimately in performing PD calculations.

According to various embodiments, the computing device uses the ML model in providing various pieces of PD information from the single image. For example, the ML model can provide PD measurements for the subject for near-viewing purposes, intermediate-viewing purposes, and far-viewing purposes. Accordingly, the ML model provides increased utility of a single image of a subject in calculating PD measurements and further provides reduced memory usage.

The various examples will be described in detail with reference to the accompanying drawings. Wherever preferable, the same reference numbers will be used throughout the drawings to refer to the same or like parts. References made throughout this disclosure relating to specific examples and implementations are provided solely for illustrative purposes but, unless indicated to the contrary, are not meant to limit all examples.

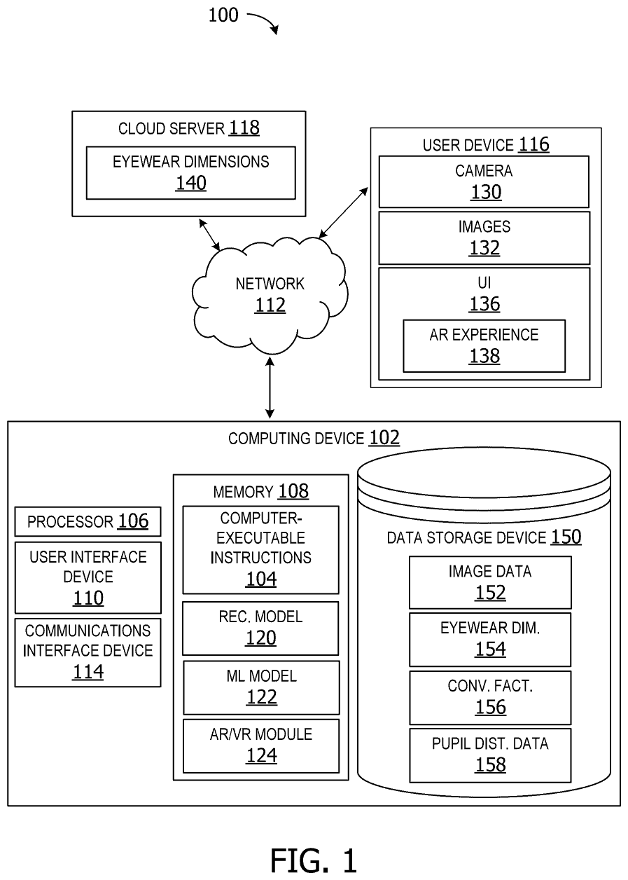

FIG. 1 illustrates an example block diagram of a system 100 for measuring pupillary distances (PD). In the example of FIG. 1, computing device 102 represents any device executing computer-executable instructions 104 (e.g., as application programs, operating system functionality, or both) to implement the operations and functionality associated with the computing device 102. Computing device 102 can represent a group of processing units or other computing devices. In some embodiments, the computing device 102 has at least one processor 106 and a memory 108. Computing device 102, in other embodiments includes a user interface device 110.

The processor 106 includes any quantity of processing units (such as a microcontroller, a microprocessor, central processing unit core(s), an ASIC, an FPGA, and/or other similar hardware device) and is suitable to execute the computer-executable instructions 104. The computer-executable instructions 104 are performed by the processor 106, performed by multiple processors within the computing device 102 or performed by a processor external to the computing device 102. In some embodiments, processor 106 is programmed to execute instructions such as those illustrated in the figures.

The computing device 102 further has one or more computer-readable media such as the memory 108. The memory 108 includes any quantity of media associated with or accessible by the computing device 102. The memory 108 in these examples is internal to computing device 102 (as shown in FIG. 1). In other embodiments, the memory 108 is external to the computing device (not shown) or both (not shown). The memory 108 can include a read-only memory and/or memory wired into an analog computing device.

The memory 108 stores data, such as one or more applications, such a recognition model 120 configured to utilize computer visional techniques to identify and/or recognize objects from images, such as images from user device 116, for example; a machine learning (ML) model 122 trained to provide PD information based on images of a subject's pupils, such as images from user device 116, for example; and an augmented reality (AR) and virtual reality (VR) module 124 configured to generate AR and/or VR experiences for gathering images of a subject's pupil via user device 116. The applications, when executed by processor 106, operate to perform functionality on the computing device 102. The applications can communicate with counterpart applications or services such as web services accessible via a network 112 or multiple communication networks 112. In an example, the applications represent downloaded client-side applications that correspond to server-side services executing in a cloud.

In other embodiments, the user interface device 110 includes a graphics card for displaying data to the user and receiving data from the user. The user interface device 110 can also include computer-executable instructions (e.g., a driver) for operating the graphics card. Further, the user interface device 110 can include a display (e.g., a touch screen display or natural user interface) and/or computer-executable instructions (e.g., a driver) for operating the display. The user interface device 110 can also include one or more of the following to provide data to the user or receive data from the user: speakers, a sound card, a camera, a microphone, a vibration motor, one or more accelerometers, a BLUETOOTH® brand communication module, wireless broadband communication (LTE) module, global positioning system (GPS) hardware, and a photoreceptive light sensor. In a non-limiting example, the user inputs commands or manipulates data by moving the computing device 102 in one or more ways.

Network 112 is implemented by one or more physical network components, such as, but without limitation, routers, switches, network interface cards (NICs), and other network devices. Network 112 is any type of network for enabling communications with remote computing devices, such as, but not limited to, a local area network (LAN), a subnet, a wide area network (WAN), a wireless (Wi-Fi) network, or any other type of network. In this example, network 112 is a WAN, such as the Internet. However, in other embodiments, network 112 is a local or private LAN.

In some embodiments, computing device 102 optionally includes a communications interface device 114. The communications interface device 114 includes a network interface card and/or computer-executable instructions (e.g., a driver) for operating the network interface card. Communication between the computing device 102 and other devices, such as but not limited to user device 116, can occur using any protocol or mechanism over any wired or wireless connection. In some embodiments, the communications interface device 114 is operable with short range communication technologies such as by using near-field communication (NFC) tags.

The user device 116 represents any device executing computer-executable instructions. User device 116 can be implemented as a mobile computing device, such as, but not limited to, a wearable computing device, a mobile telephone, laptop, tablet, computing pad, netbook, gaming device, and/or any other portable device. The user device 116 includes at least one processor and a memory. The user device can include a camera 130 configured to gather image data used by computing device 102. Camera 130 can store and/or capture images 132 utilized by computing device 102 for determining PDs. User device 116 includes a user interface 136, which may be substantially the same as user interface device 110 previously discussed. AR/VR module 124 can display to the user an interactive AR experience 138 on user interface 136.

The cloud server 118 is a logical server providing services to the computing device 102 or other clients, such as, but not limited to, the user device 116. Cloud server 118 is hosted and/or delivered via network 112. In some non-limiting examples, cloud server 118 is associated with one or more physical servers in one or more data centers. In other embodiments, the cloud server 118 is associated with a distributed network of servers. Cloud server 118 can store eyewear dimensions 140 that can be retrieved by computing device 102 based on eyewear detected from an image 132 by recognition model 120.

The system 100 can optionally include a data storage device 150 for storing data, such as, but not limited to, image data 152 from user device 116; eyewear dimensions 154 retrieved from cloud server 118; pixel-to-millimeter conversion factors 156 calculated by computing device 102 based on eyewear dimensions 140; and pupil distance data 158 measured by computing device 102 based on images 132. The data storage device 150 can include one or more different types of data storage devices, such as, for example, one or more rotating disks drives, one or more solid state drives (SSDs), and/or any other type of data storage device. The data storage device 150 in some non-limiting examples includes a redundant array of independent disks (RAID) array. In some non-limiting examples, the data storage device(s) provide a shared data store accessible by two or more hosts in a cluster. For example, the data storage device can include a hard disk, a redundant array of independent disks (RAID), a flash memory drive, a storage area network (SAN), or other data storage device. In other embodiments, the data storage device 150 includes a database.

The data storage device 150 in this example is included within the computing device 102, attached to the computing device, plugged into the computing device, or otherwise associated with the computing device 102. In other embodiments, data storage device 150 includes a remote data storage accessed by the computing device via network 112, such as a remote data storage device, a data storage in a remote data center, or a cloud storage. Similarly, although recognition model 120, ML model 122, and AR/VR module 124 are depicted within computing device 102, according to various embodiments, these applications can be implemented on a remote or cloud memory storage.

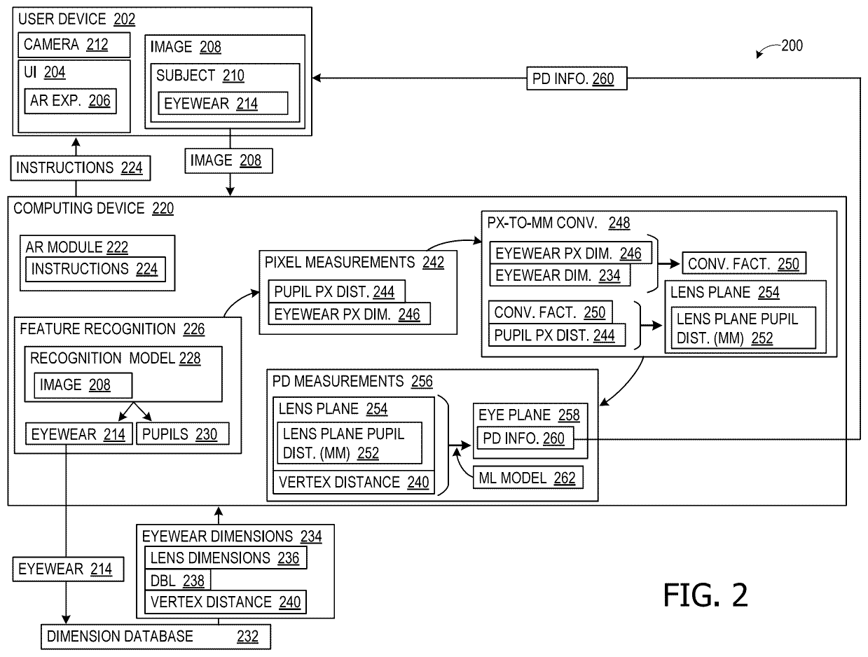

FIG. 2 is a block diagram illustrating an example system 200, which may be substantially similar in many respects to the system 100, for determining pupillary distances (PD). Those with skill in the art will recognize that various elements of system 200 correspond with elements of system 100 previously discussed. System 200 includes a user device 202 accessed by a subject 210 for measuring the PD of subject 210. User device 202 includes a user interface (UI) 204 that displays an augmented reality (AR) experience 206 for guiding subject 210 through the process of generating PD measurements. As those with skill in the art will understand, AR experiences, such as AR experience 206, combine real world elements captured by a camera with virtual elements. Specifically, the AR experience 206 provides instructions for capturing an image 208 of the subject's 210 face via camera 212 while the subject 210 is wearing eyewear 214. Eyewear 214 can comprise any of various types of eyewear, such as eyeglasses, for example. Image 208 is then delivered to a computing device 220.

As shown, computing device 220 includes AR module 222 that generates instructions 224 that are received by user device 202 and used as part of AR experience 206. For example, instructions 224 can specify a desired distance or location of subject 210 relative to camera 212 to ensure image 208 is captured with subject 210 positioned at a proper location and/or orientation for computing device 220 to perform PD measurements. AR module 222 can analyze live streaming data from camera 212 and provide instructions accordingly. For example, the instructions 224 can instruct subject 210 to move toward/away camera 212, or to change the orientation of their head based on a detected location of subject 210 within the streaming data from camera 212. In some embodiments, AR module 222 can automatically capture image 208 in response to determining that subject 210 is wearing eyewear 214, in the proper position, and at the proper orientation, for example. While instructions 224 are described herein as being part of an AR experience 206, those with skill in the art will understand that various embodiments fall within the scope of this disclosure. For example, instructions 224 can simply direct the subject 210 or a practitioner in how to position subject 210 relative to camera 212.

Computing device 220 can perform feature recognition 226 on image 208 using a recognition model 228. Recognition model 228 can be a machine learning model, such as a deep learning model, trained to recognize and/or identify various features related to image 208, such as eyewear 214 and the various elements thereof, and pupils 230 of subject 210 using any of various computer vision applications and techniques. In some embodiments, recognition model 228 comprises a deep convolutional neural network (CNN) that is fine-tuned on a dataset to optimize performance for a given task, such as identifying and recognizing eyewear 214, for example. In some embodiments, the CNN comprises ResNet-50, for example. In some embodiments, recognition model 228 comprises a feature pyramid network (FPN) deep learning architecture. The recognition model 228 enables strong multi-scale feature extraction that is particularly effective at recognizing objects of varying sizes and shapes. Eyewear 214 can comprise any of various eye-related accessories, such as prescription eyeglasses, reading glasses, non-prescription eyeglasses, sunglasses, smart glasses, and any of various other eye-related accessories worn by a subject near the eyes. While some example recognition models 228 are described, those with skill in the art will understand any of various computer vision models and architectures are employed as part of recognition mode 228 according to various embodiments of this disclosure.

As part of feature recognition 226, computing device 220 recognizes eyewear 214 included in image 208 and, based on the recognition, references dimension database 232 to determine various eyewear dimensions 234 associated with the eyewear 214. For example, eyewear dimensions 234 can include lens dimensions 236 related to various dimensions of lenses of eyewear 214. Eyewear dimensions 234 can include the distance between the lenses (DBL) 238 of eyewear 214, which can also be referred to as bridge distance for eyewear 214. Eyewear dimensions 234, in other examples, includes a vertex distance 240 associated with eyewear 214, which, as those with skill in the art will understand, is the distance between a plane associated with the lenses of eyewear 214 and plane associated with the front of the eyes of the user. Eyewear dimensions 234 can be defined values associated with eyewear 214 and stored in database 232. For example, eyewear dimensions 234 can come from product specification information associated with eyewear 214 and can be expressed using a standard unit of measure, such as millimeters, for example.

Computing device 220 can further perform pixel measurements 242 for features identified via recognition model 228. For example, pixel measurements 242 can include determining the distance in pixels (herein referred to as “pixel distance”) between the pupils 230 identified by recognition model 228, as represented by pupil pixel distance 244. Pixel measurements 242 can further include measuring, in pixels, dimensions of various features of eyewear 214 identified by recognition model 228, as represented by eyewear pixel dimensions 246. Those with skilled in the art will understand the pixel measurements 242 can be performed using any of various computer vision technologies software and/or techniques.

Computing device 220 can then perform pixel-to-millimeter conversions 248 to convert various distances determined in pixels to millimeters. Specifically, in performing this conversion, pixel-to-millimeter conversions 248 incorporates eyewear dimensions 234 as a reference. For example, eyewear pixel dimensions 246 are compared to known associated values found in eyewear dimensions 234, which can be in millimeters, for example. Accordingly, from this, a pixel-to-millimeter conversion factor 250 can be determined that can be used to convert a value measured in pixels from image 208 to millimeters.

While eyewear dimensions 234 and various measurements are disclosed in illustrative examples of this disclosure as having millimeters as a unit of measure, various units of measure can be used for eyewear dimensions 234 and associated measurements according to various embodiments of this disclosure.

Pixel-to-millimeter conversion factor 250 can then be used to determine the distance between the pupils in millimeters. As shown, the pixel-to-millimeter conversion factor 250 is applied to pupil pixel distance 244 to calculate a lens plane pupil distance 252. Since pixel-to-millimeter conversion factor 250 is generated using eyewear dimensions 234 as a reference, and specifically uses dimensions associated with lenses of the eyewear 214, the resulting measurements relate to a measurements disposed on a lens plane 254 associated with the lenses of eyewear 214. The pupils are not disposed on lens plane 254, but are recessed from lens plane 254 by the vertex distance 240. Thus, pixel-to-millimeter conversions 248 do not calculate the actual distance between the pupils, but instead the lens plane pupil distance 252 of pupils projected on lens plane 254.

Computing device 220 then performs PD measurements 256 to determine the actual pupillary distance information for subject 210. PD measurements 256 apply vertex distance 240 to lens plane pupil distance 252 to determine various pieces of PD information 260 related to the distance between the pupils on an eye plane 258 associated with the eyes of subject 210. Thus, PD information 260 relates to the actual PD for the subject 210 as measured from the surface of the eyes (eye plane 258). In some embodiments, as will be discussed in greater detail below, a machine learning (ML) model 262 is used in generating the PD information 260. PD information 260 can then be delivered as an output of computing device 220, such as to user device 202, as shown, so that the PD information 260 can be used by subject 210 or a practitioner for any of various ophthalmological and/or optometric purposes.

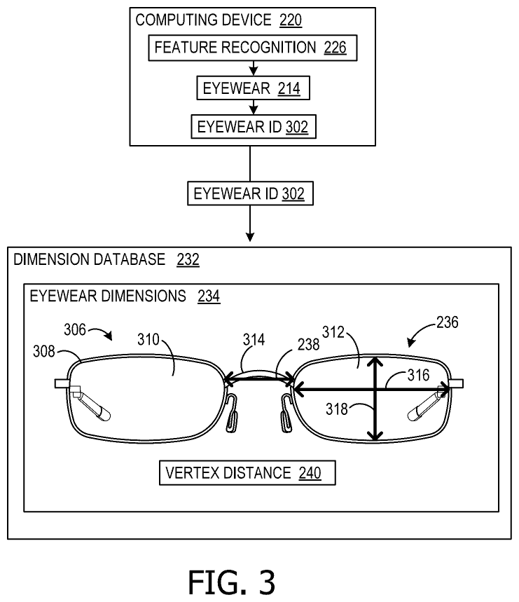

FIG. 3 is a block diagram illustrating dimension retrieval of eyewear dimensions 234 from dimension database 232 according to an example. During feature recognition 226, recognition model 228 is trained to recognize various pieces of eyewear based on recognizable features associated with the eyewear, such as a size, shape, and/or form factor of the eyewear, for example. Upon recognizing the eyewear in image 208 as eyewear 214, computing device 220 determines an eyewear identifier (ID) 302 associated with eyewear 214. For example, eyewear ID 302 can be a model number, serial number, or any other unique ID associated with eyewear 214.

Computing device 220 can then reference dimension database 232 to find eyewear dimensions 234 associated with eyewear 214 using eyewear ID 302. As shown, eyewear 214 is a pair of eyeglasses 306 including a frame 308 holding a first lens 310 and a second lens 312, and including a nose bridge 314. As those with skill in the art will recognize, according to various embodiment lens 310 and lens 312 are mirror-images of each other in terms of size and shape. Thus, according to some embodiments, lens 310 and lens 312 have matching dimensions, such as matching heights and widths, for example.

Eyewear dimensions 234 include various dimensions related to eyeglasses 306. Eyewear dimensions 234 can include a lens width 316, which is an overall width of each lens 310, 312. Eyewear dimensions 234 can include a lens height 318, which is an overall height of each lens 310, 312. As those with skill in the art will understand, according to various embodiments, such as the embodiments discussed herein, the lens width 316 for lens 312 is the same as the lens width 316 for lens 310, and the lens height 318 for lens 310 is the same as the lens height 318 for lens 312. However, in other embodiments, the lens width 316 and lens height 318 between lens 310 and lens 312 can differ from each other. Also included in eyewear dimensions 234 are the DBL 238 and vertex distance 240.

As previously discussed, the eyewear dimensions 234, such as lens width 316, lens height 318, DBL 238 (also referred to herein as the bridge distance), and vertex distance 240 can be defined according to manufacturing or product specifications associated with eyeglasses 306. In some embodiments, the eyewear dimensions 234 are in a standard unit of measure, such as in millimeters, for example.

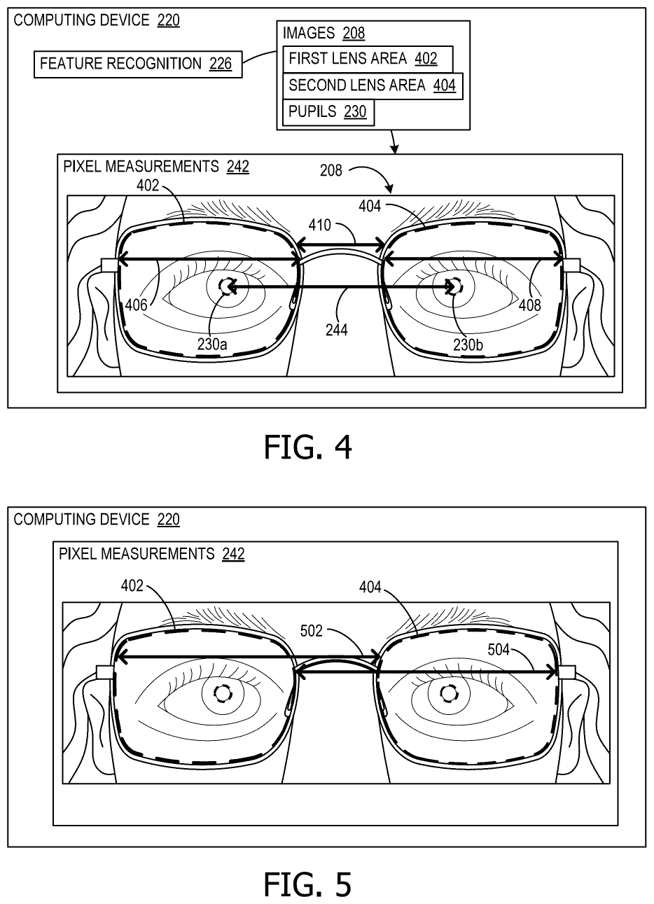

FIG. 4 illustrates how computing device 220 performs pixel measurements 242 according to an example. As shown, recognition model 228 employed during feature recognition 226 is trained to identify various features associated with image 208, such as pupils 230 and eyewear 214, as previously discussed. As part of recognizing eyewear 214, feature recognition 226 further comprises recognizing various features associated with eyewear 214, such as a first lens area 402 associated with lens 310 and a second lens area 404 associated with lens 312.

With the features recognized and their position identified within image 208 via feature recognition 226, pixel measurements 242 can be performed to measure distances between various features in pixels. As shown, the pupil pixel distance 244 can be measured between recognized pupil 230a and pupil 230b. Various eyewear pixel dimensions 246 can also be measured, such as a first lens width 406 of first lens area 402, a second lens width 408 second lens area 404, and a spanning distance 410 measuring distance between the first lens area 402 and the second lens area 404.

In some embodiments, pixel measurements 242 performed in FIG. 4 may be sufficient for correlating against known eyewear dimensions 234 in performing pixel-to-millimeter conversions 248. However, in some embodiments, accuracy may be improved when additional pixel measurements are performed.

FIG. 5 shows additional pixel measurements 242 that can be measured by computing device 220 to improve accuracy of pixel-to-millimeter conversions 248 according to an example. Pixel measurements 242 can include a first width 502, which is a width measuring a distance, in pixels, from an outer edge of first lens area 402 to an inner edge of second lens area 404. Pixel measurements 242 can further include a second width 504 measuring a distance, in pixels, from an outer edge of second lens area 404 to an inner edge of first width 502.

As will become clearer below, the use of first width 502 and second width 504 provide increased accuracy in pixel-to-millimeter conversions 248 in addition to conversions based on eyewear pixel dimensions 246. For example, in some embodiments, recognition model 228 may be unable to detect the exact boundaries of lens 310 and lens 312 when generating first lens area 402 and second lens area 404 due to frame 308 overlapping or covering parts of lens 310, 312. Accuracy is improved in correlating measured pixel lengths to known eyewear dimensions 234 when first width 502 and second width 504 are used in performing pixel-to-millimeter conversions 248, as will be discussed in greater detail below.

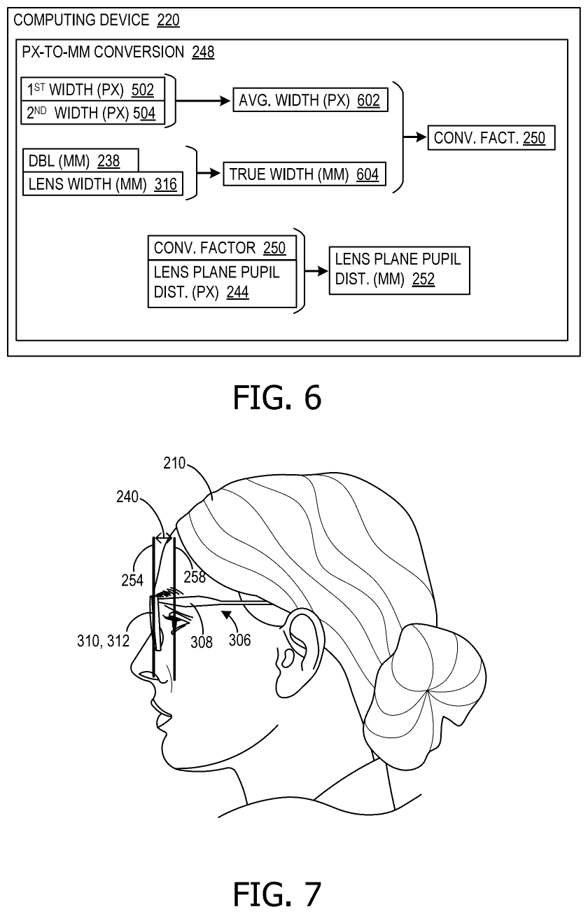

FIG. 6 is a block diagram illustrating pixel-to-millimeter conversions 248 performed by computing device 220 according to an example. While FIG. 6 depicts one illustrative example for performing pixel-to-millimeter conversions 248, those with skill in the art will recognize that various other calculations can be performed as part of pixel-to-millimeter conversions 248 according to various embodiments of this disclosure. As shown, computing device 220 averages first width 502 and second width 504 to determine an average width 602. In some embodiments, average width 602 is calculated for pixel-to-millimeter conversions 248 rather than just using one of either first width 502 or second width 504 for various reasons. For example, the eyeglasses 306 may be oriented at a slight angle relative to camera 212, or the user's head might be slightly turned relative to camera 212 such that first width first width 502 and second width 504 are different from each other. Thus, such discrepancies caused by orientations related to subject 210 are alleviated by finding average width 602 based on first width 502 and second width 504.

Pixel-to-millimeter conversions 248 further includes adding DBL 238 to lens width 316 from eyewear dimensions 234 to determine a true width 604. As those with skill in the art understand, true width 604 and average width 602 may be a measure of a same section of eyeglasses 306, just in different units of measure. Average width 602 is a measurement, in pixels, from an outer edge of one lens 310, 312 to an inner edge of the other lens 310, 312, which is the same as DBL 238 added to lens width 316 (true width 604). Thus, by correlating true width 604 (in millimeters) and average width 602 (in pixels), pixel-to-millimeter conversion factor 250 can be generated which assigns a millimeter value for each pixel, or vice versa.

Pixel-to-millimeter conversion factor 250 can then be applied to the pupil pixel distance 244 to determine the lens plane pupil distance 252, which is the distance between the pupils 230 in millimeters as projected on the lens plane 254, as previously discussed.

As mentioned, FIG. 6 depicts just one example of pixel-to-millimeter conversions 248. In other embodiments of this disclosure, pixel-to-millimeter conversions 248 is performed using other eyewear pixel dimensions 246, such as first lens width 406, second lens width 408, and/or spanning distance 410, for example. However, as previously discussed, using first width 502 and second width 504 provides advantages in accuracy, as such dimensions are not influenced by orientations or angles of the subject 210 relative to camera 212 and account for undetectable portions of lenses 310, 312 due to frame 308.

As those with skill in the art will recognize, lens plane pupil distance 252 is not the actual PD of the patient, as the lens plane pupil distance 252 measures the distance between representations of the pupils 230 projected on lens plane 254. FIG. 7 illustrates a side profile subject 210 so that lens plane 254 and eye plane 258 can be better understood. As shown, lens plane 254 is the plane on which lenses 310, 312 are positioned. Eye plane 258 is parallel to lens plane 254 and is a plane contacting the cornea of both of subject's 210 eyes. The PD for a subject 210, even in manual PD measurements, can be said to be measured on eye plane 258.

In some embodiments, lens plane 254 and eye plane 258 are parallel to each other and separated by vertex distance 240. As previously discussed, in some embodiments, vertex distance 240 is provided to computing device 220 as part of eyewear dimensions 234. However, those with skill in the art will understand that other embodiments are included in this disclosure. For example, the vertex distance 240 in some examples is a standard value programmed to computing device 220; in some examples is a measured value input into computing device 220 by a practitioner assisting subject 210; or in some examples is a calculated value, such as one calculated by computing device 220 based on various input data, such as provided or detected size information related to facial features of subject 210 and/or eyewear 214, for example.

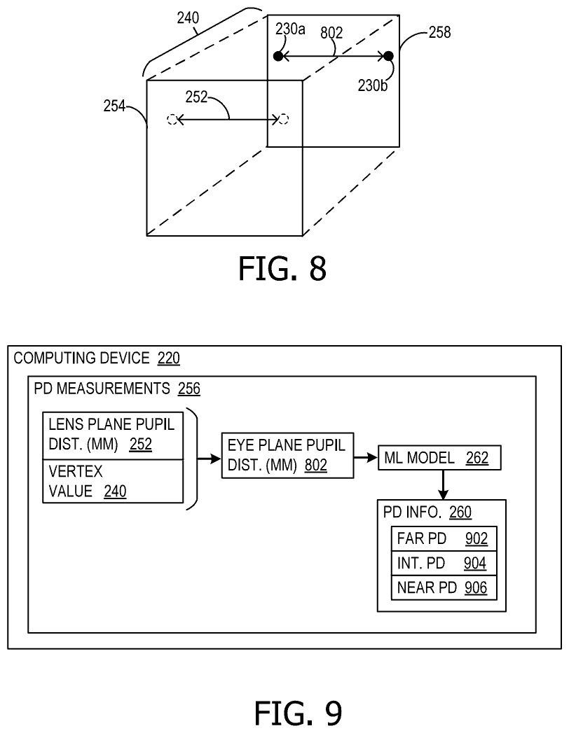

FIG. 8 further explains the relationship between lens plane 254 and eye plane 258. Lens plane pupil distance 252 is a measurement of a projection of pupils 230a, 230b on lens plane 254. However, the lens plane pupil distance 252 is not the actual distance between the pupils, which is represented by eye plane pupil distance 802, as lens plane 254 is displaced from eye plane 258 by the vertex distance 240. Thus, lens plane pupil distance 252 will actually be less than eye plane pupil distance 802 because pupils 230a, 230b, are further away from camera 212 than lens plane 254.

FIG. 9 is a block diagram illustrating example PD measurements 256 performed by computing device 220. With the known values of lens plane pupil distance 252 and vertex distance 240, eye plane pupil distance 802 can be calculated by computing device 220 using trigonometry and/or other mathematics principles.

However, according to some embodiments, eye plane pupil distance 802 may not directly correspond to the PD of the subject 210. The PD of a person varies depending on what their eyes are focused on when the PD is being measured. When a subject is looking at something nearby, such as when reading a book, their PD is shorter than when they are looking at something further away. That is, a subject's PD increases as they look further off into the distance. So, while eye plane pupil distance 802 may reflect the current PD of the 210 when image 208 is captured, it may not reflect PD desired for various ophthalmological and/or optometric purposes.

PDs can be classified according to any of a number of different classifications, such as a far PD 902 corresponding to a PD for subject 210 viewing an object faraway at a first distance farthest from the subject; an intermediate PD 904 corresponding to a PD for subject 210 viewing an object at an intermediate distance at a second distance closer to the subject than the first distance; and a near PD 906 corresponding to a PD for subject 210 viewing an object nearby at a third distances closer to the subject than the first distance and the second distance. Those with skill in the art will recognize how these different PDs can be utilized by a practitioner in providing ophthalmological and/or optometric care. For example, far PD 902 could be used for purposes related to subject's 210 ability to see at far distances, such as for driving a car, for example. Intermediate PD 904 could be used for purposes related to subject's 210 ability to see at intermediate distances, such as for watching television, for example. Near PD 906 could be used for purposes related to subject's 210 ability to see at near distances, such as for reading, for example. Additionally, all PD distances 902-906 could be used to provide variable optical powers in a pair of eyeglasses or prescriptive lenses to provide the subject 210 with proper optical power according to the task being performed. Often, the far PD 902 is considered a subject's true PD for various ophthalmological and/or optometric purposes.

To obtain each of the PD distances 902-906 from a single image 208, computing device 220 can utilize ML model 262. For example, ML model 262 can be trained with images taken according to the same or similar instructions as instructions 224 for various patients, and the PD for the patients can be taken using a pupilometer to measure their actual PD for PD distances 902-906. Thus, ML model 262 can be trained to provide PD information 260, including far PD 902, intermediate PD 904, and near PD 906, when provided with eye plane pupil distance 802.

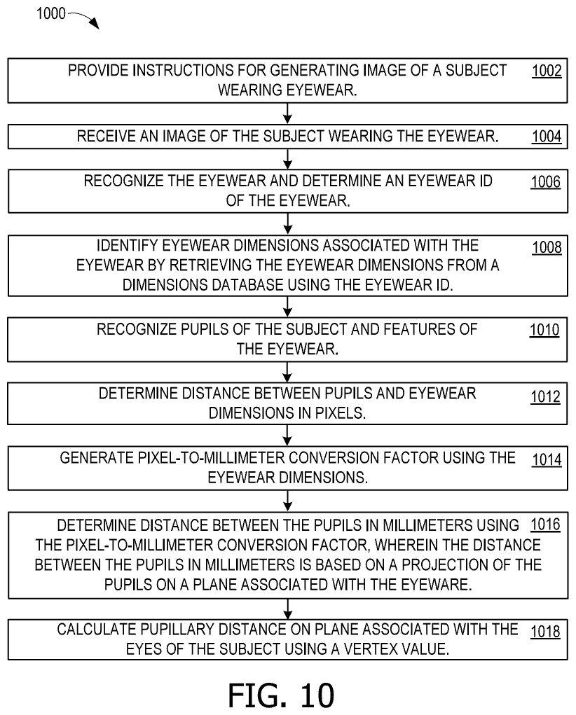

FIG. 10 is a flowchart illustrating an example method 1000 for determining the PD of a subject 210. Method 1000 can start ant block 1002 by providing instructions 224 for capturing image 208 of subject 210 wearing eyewear 214, such as via an AR experience 206. Method 1000 can continue at block 1004 by computing device 220 receiving image 208 from user device 202. Method 1000 can continue at block 1006 by performing feature recognition 226 via recognition model 228 and recognizing eyewear 214 from image 208 and further determining an eyewear ID 302 associated with the recognized eyewear 214. Method 1000 can continue to block 1008 by identifying eyewear dimensions 234 associated with the eyewear ID 302 by retrieving eyewear dimensions 234 from dimension database 232.

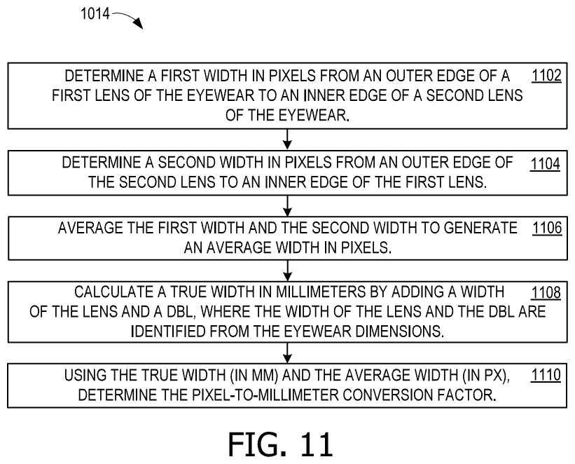

Method 1000 can continue to block 1010 by performing feature recognition 226 via recognition model 228 and recognizing pupils 230 of subject 210. Method 1000 can continue to block 1012 by performing pixel measurements 242 and determining pupil pixel distance 244 and eyewear pixel dimensions 246. Method 1000 can continue to block 1014 wherein computing device 220 performs pixel-to-millimeter conversions 248 to ultimately generate a pixel-to-millimeter conversion factor 250, which is discussed in greater detail in FIG. 11.

Method 1000 can continue to block 1016 where pixel-to-millimeter conversions 248 is continued by computing device 220 and pixel-to-millimeter conversion factor 250 is used to convert pupil pixel distance 244 to lens plane pupil distance 252. Method 1000 can continue to block 1018 where computing device 220 performs PD measurements 256, which can include using vertex distance 240 to determine eye plane pupil distance 802 and further using ML model 262 to determine PD information 260.

While method 1000 is shown with blocks 1002-1018 occurring in a certain order, those with skill in the art will understand that blocks 1002-1018 can be performed according to any of various orders without departing from the scope of this disclosure. Additionally, method 1000 can include more or fewer than blocks 1002-1018 without departing from the scope of this disclosure.

FIG. 11 is a flowchart illustrating a method for performing pixel-to-millimeter conversions 248 to generate pixel-to-millimeter conversion factor 250, such as for performing block 1014 of FIG. 10. The method can start at block 1102 by computing device 220 performing pixel measurements 242 to determine first width 502. The method can continue to block 1104 by computing device 220 performing pixel measurements 242 to determine second width 504. The method can continue to block 1106 by computing device 220 determining average width 602 based on first width 502 and second width 504. The method can continue to block 1108 by computing device 220 calculating a true width 604 by adding DBL 238 and lens width 316. The method can continue to block 1110 where computing device 220 correlates average width 602 and true width 604 to determine pixel-to-millimeter conversion factor 250.

As has been previously discussed, FIG. 11 is just one of various methods of this disclosure that can be employed for performing pixel-to-millimeter conversion 248 to generate pixel-to-millimeter conversion factor 250. While the method of block 1014 is shown with blocks 1102-1110 occurring in a certain order, those with skill in the art will understand that blocks 1102-1110 can be performed according to any of various orders without departing from the scope of this disclosure. Additionally, the method of block 1014 can include more or fewer than blocks 1102-1110 without departing from the scope of this disclosure.

### Additional Examples

In an example scenario, the systems herein (such as system 100 and 200, for example) are utilized by ophthalmological and/or optometric providers, such as for providing eyecare care and/or corrective lenses consultations to their patients. The system provides AR instructions for capturing an image of a subject patient wearing eyewear that can be used by the system as a reference for determining the PD of the subject. The system identifies dimensions of eyewear worn by a subject from the image and detects, from the image, a first pupil and a second pupil of the subject. The system determines a pixel distance between the first pupil and the second pupil and converts the pixel distance to a measured distance using the eyewear dimensions, wherein the measured distance is on a lens plane associated with lenses of the eyewear. The computing device determines a location of an eye plane associated with the eyes of the subject relative to the position of the lens plane using a vertex value and calculates a PD for the subject using the measured distance on the lens plane, wherein the calculated PD is the distance between the first pupil and the second pupil on the eye plane

Alternatively, or in addition to the other examples described herein, examples include any combination of the following:
recognizing the eyewear using a recognition model; determining an eyewear identifier associated with the recognized eyewear; and retrieving the eyewear dimensions from a dimensions database using the eyewear identifier;
determining, from the image, a first width from an outer edge of the first lens to an inner edge of the second lens in pixels; determining, from the image, a second width from an outer edge of the second lens to an inner edge of the first lens in pixels; averaging the first width and the second width to determine an average width in pixels; determining a true width in millimeters by adding the bridge distance to one of the first dimension or the second dimension; and performing pixel-to-millimeter conversion using the true width and the average width to determine the measured distance between the first pupil and the second pupil;
utilizing a machine learning (ML) model to determine: a far PD corresponding to a PD for the subject when viewing an object at a faraway distance; an intermediate PD corresponding to a PD for the subject when viewing an object at an intermediate distance; and a near PD corresponding to a PD for the subject when viewing an object at a near distance;
providing, to a user device, instructions for positioning the subject relative to a camera associated with the user device; and receiving the image from a user device, wherein the image is generated by the camera; and
where the instructions are provided to the user device as part of an augmented reality (AR) experience performed via the user device for determining the calculated PD.

At least a portion of the functionality of the various elements in FIG. 1 can be performed by other elements in FIG. 1, or an entity (e.g., processor 106, web service, server, application program, computing device, etc.) not shown in FIG. 1.

In some examples, the operations illustrated in FIGS. 10 and 11 can be implemented as software instructions encoded on a computer-readable medium, in hardware programmed or designed to perform the operations, or both. For example, aspects of the disclosure can be implemented as a system on a chip or other circuitry including a plurality of interconnected, electrically conductive elements.

While the aspects of the disclosure have been described in terms of various examples with their associated operations, a person skilled in the art would appreciate that a combination of operations from any number of different examples is also within scope of the aspects of the disclosure.

The term “Wi-Fi” as used herein refers, in some examples, to a wireless local area network using high frequency radio signals for the transmission of data. The term “BLUETOOTH®” as used herein refers, in some examples, to a wireless technology standard for exchanging data over short distances using short wavelength radio transmission. The term “NFC” as used herein refers, in some examples, to a short-range high frequency wireless communication technology for the exchange of data over short distances.

### Example Operating Environment

Although described in connection with an example computing device 102, 220, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, augmented reality (AR) devices, mixed reality devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions, or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable, and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals per se. Example computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. In contrast, communication media typically embody computer readable instructions, data structures, program modules, or the like in a modulated data signal such as a carrier wave or other transport mechanism and include any information delivery media.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A method for determining pupillary distances, the method comprising:
detecting eyewear worn by a subject of an image;
identifying eyewear dimensions of the eyewear including a first dimension of a first lens of the eyewear, a second dimension of a second lens of the eyewear, and a bridge distance measuring a distance between the first lens and the second lens;
detecting, from the image, a first pupil and a second pupil of the subject;
determining, from the image a pixel distance between the first pupil and the second pupil;
converting the pixel distance to a measured distance using the eyewear dimensions, wherein the measured distance is on a lens plane associated with the first lens and the second lens;
determining a location of an eye plane associated with the eyes of the subject relative to a position of the lens plane using a vertex value; and
calculating a pupillary distance (PD) using the measured distance on the lens plane, wherein the calculated PD is the distance between the first pupil and the second pupil on the eye plane.

2. The method of claim 1, further comprising:
recognizing the eyewear using a recognition model;
determining an eyewear identifier associated with the recognized eyewear; and
retrieving the eyewear dimensions from a dimensions database using the eyewear identifier.

3. The method of claim 1, wherein the first dimension is a width of the first lens in millimeters, the second dimension is a width of the second lens in millimeters, and the bridge distance is in millimeters.

4. The method of claim 3, further comprising:
determining, from the image, a first width from an outer edge of the first lens to an inner edge of the second lens in pixels;
determining, from the image, a second width from an outer edge of the second lens to an inner edge of the first lens in pixels;
averaging the first width and the second width to determine an average width in pixels;
determining a true width in millimeters by adding the bridge distance to one of the first dimension or the second dimension; and
performing pixel-to-millimeter conversion using the true width and the average width to determine the measured distance between the first pupil and the second pupil.

5. The method of claim 1, wherein calculating the PD comprises utilizing a machine learning (ML) model to determine:
a far PD corresponding to a PD for the subject when viewing an object at a faraway distance;
an intermediate PD corresponding to a PD for the subject when viewing an object at an intermediate distance; and
a near PD corresponding to a PD for the subject when viewing an object at a near distance,
wherein the calculated PD comprises the far PD.

6. The method of claim 1, further comprising:
providing, to a user device, instructions for positioning the subject relative to a camera associated with the user device; and
receiving the image from a user device, wherein the image is generated by the camera.

7. The method of claim 6, wherein the instructions are provided to the user device as part of an augmented reality (AR) experience performed via the user device for determining the calculated PD.

8. A system for determining pupillary distances, the system comprising:
a processor; and
a computer-readable medium storing instructions that are operative upon execution by the processor to:
detect eyewear worn by a subject of an image;
identify eyewear dimensions of the eyewear including a first dimension of a first lens of the eyewear, a second dimension of a second lens of the eyewear, and a bridge distance measuring a distance between the first lens and the second lens;
detect, from the image, a first pupil and a second pupil of the subject;
determine, from the image, a pixel distance between the first pupil and the second pupil;
convert the pixel distance to a measured distance using the eyewear dimensions, wherein the measured distance is on a lens plane associated with the first lens and the second lens;
determine a location of an eye plane associated with the eyes of the subject relative to a position of the lens plane using a vertex value; and
calculate a pupillary distance (PD) using the measured distance on the lens plane, wherein the calculated PD is the distance between the first pupil and the second pupil on the eye plane.

9. The system of claim 8, wherein the computer-readable medium further stores instructions operative by the processor to:
recognize the eyewear using a recognition model;
determine an eyewear identifier associated with the recognized eyewear; and
retrieve the eyewear dimensions from a dimensions database using the eyewear identifier.

10. The system of claim 8, wherein the first dimension is a width of the first lens in millimeters, the second dimension is a width of the second lens in millimeters, and the bridge distance is in millimeters.

11. The system of claim 10, wherein the computer-readable medium further stores instructions operative by the processor to:
determine, from the image, a first width from an outer edge of the first lens to an inner edge of the second lens in pixels;
determine, from the image, a second width from an outer edge of the second lens to an inner edge of the first lens in pixels;
average the first width and the second width to determine an average width in pixels;
determine a true width in millimeters by adding the bridge distance to one of the first dimension or the second dimension; and
perform pixel-to-millimeter conversion using the true width and the average width to determine the measured distance between the first pupil and the second pupil.

12. The system of claim 8, wherein calculating the PD comprises utilizing a machine learning (ML) model to determine:
a far PD corresponding to a PD for the subject when viewing an object at a faraway distance;
an intermediate PD corresponding to a PD for the subject when viewing an object at an intermediate distance; and
a near PD corresponding to a PD for the subject when viewing an object at a near distance,
wherein the calculated PD comprises the far PD.

13. The system of claim 8, wherein the computer-readable medium further stores instructions operative by the processor to:
provide, to a user device, instructions for positioning the subject relative to a camera associated with the user device; and
receive the image from a user device, wherein the image is generated by the camera.

14. The system of claim 13, wherein the instructions are provided to the user device as part of an augmented reality (AR) experience performed via the user device for determining the calculated PD.

15. A computer-readable medium storing instructions for determining pupillary distances, the instructions operative upon execution by a processor to:
detect eyewear worn by a subject of an image;
identify eyewear dimensions of the eyewear including a first dimension of a first lens of the eyewear, a second dimension of a second lens of the eyewear, and a bridge distance measuring a distance between the first lens and the second lens;
detect, from the image, a first pupil and a second pupil of the subject;
determine, from the image, a pixel distance between the first pupil and the second pupil;
convert the pixel distance to a measured distance using the eyewear dimensions, wherein the measured distance is on a lens plane associated with the first lens and the second lens;
determine a location of an eye plane associated with the eyes of the subject relative to a position of the lens plane using a vertex value; and
calculate a pupillary distance (PD) using the measured distance on the lens plane, wherein the calculated PD is the distance between the first pupil and the second pupil on the eye plane.

16. The computer-readable medium of claim 15, wherein the computer-readable medium further stores instructions operative by the processor to:
recognize the eyewear using a recognition model;
determine an eyewear identifier associated with the recognized eyewear; and
retrieve the eyewear dimensions from a dimensions database using the eyewear identifier.

17. The computer-readable medium of claim 15, wherein the first dimension is a width of the first lens in millimeters, the second dimension is a width of the second lens in millimeters, and the bridge distance is in millimeters.

18. The computer-readable medium of claim 17, wherein the computer-readable medium further stores instructions operative by the processor to:
determine, from the image, a first width from an outer edge of the first lens to an inner edge of the second lens in pixels;
determine, from the image, a second width from an outer edge of the second lens to an inner edge of the first lens in pixels;
average the first width and the second width to determine an average width in pixels;
determine a true width in millimeters by adding the bridge distance to one of the first dimension or the second dimension; and
perform pixel-to-millimeter conversion using the true width and the average width to determine the measured distance between the first pupil and the second pupil.

19. The computer-readable medium of claim 15, wherein calculating the PD comprises utilizing a machine learning (ML) model to determine:
a far PD corresponding to a PD for the subject when viewing an object at a faraway distance;
an intermediate PD corresponding to a PD for the subject when viewing an object at an intermediate distance; and
a near PD corresponding to a PD for the subject when viewing an object at a near distance,
wherein the calculated PD comprises the far PD.

20. The computer-readable medium of claim 15, wherein the computer-readable medium further stores instructions operative by the processor to:
provide, to a user device, instructions for positioning the subject relative to a camera associated with the user device; and
receive the image from a user device, wherein the image is generated by the camera.
